(19) United States
(12) Patent Application Publication
(10) Pub. No.: US 2015/0164841 A1
(43) Pub. Date: Jun. 18, 2015

(54) CONCENTRATION OF OMEGA-3 POLYUNSATURATED FATTY ACIDS IN KRILL OIL

(71) Applicant: AKER BIOMARINE ANTARCTIC AS, Stamsund (NO)

(72) Inventors: Nils Hoem, Oslo (NO); Snorre Tilseth, Bergen (NO)

(21) Appl. No.: 14/413,858

(22) PCT Filed: Jul. 8, 2013
(86) PCT No.: PCT/IB2013/001959

§ 371 Date: Jan. 9, 2015

Related U.S. Application Data
(60) Provisional application No. 61/672,348, filed on Jul. 17, 2012.

(51) Int. Cl. A61K 31/202; A61K 31/201; A61K 31/122

(52) U.S. Cl. CPC A61K 31/202; A61K 31/122; A61K 31/201

(57) ABSTRACT

The present invention relates to krill oil, and in particular to krill oil with elevated levels of omega-3 fatty acids and decreased levels of saturated fatty acids.

### FIELD OF THE INVENTION

The present invention relates to krill oil, and in particular to krill oil with elevated levels of omega-3 fatty acids and decreased levels of saturated fatty acids.

### BACKGROUND OF THE INVENTION

Fish oils that are abundant in omega-3 polyunsaturated fatty acids (PUFA) have traditionally been used as the raw material for preparation of omega-3 PUFA concentrate. Since fish oils are complex mixtures of triglycerides containing fatty acids with varying chain lengths and degrees of unsaturation, separation of individual fatty acids is difficult for production of concentrated omega-3 components. Therefore, commercial production of marine oil concentrates with enhanced percentages of EPA and DHA has been a major challenge for food scientists and biotechnologists engaged in research in this area.

Methods for concentration of omega-3 PUFA are numerous, but only few are suitable for large-scale production. Distillation has been used for partial separation of mixtures of fatty acid esters. This method takes advantage of differences in the boiling point and molecular weight of fatty acids under reduced pressure. This technique requires high temperatures of approximately 250 C. Berger, R. and McPherson, W. (1979) ‘Fractional Distillation’ in J. Am. Oil Chem. Soc. 56, 743A-746A. Short-path distillation or molecular distillation uses lower temperatures and short heating intervals. However, fractionation of fish oil esters is difficult since separation of these components becomes less effective with increasing molecular weight. Weitkamp, A. W. (1955) ‘Distillation’ in J. Am. Oil Chem. Soc. 32, 640-646; Brevik, H. (1992) ‘N-3 Concentrates: A Scandinavian View-point’ in AOCS Short Courses, Modern Application of Marine Oils, 7-8 May, Toronto, ON, Canada.

The most widely used distillation procedure is fractional distillation of methyl esters under reduced pressure (0.1-1.0 mmHg). Even under these conditions, moderately high temperatures are required; the more highly unsaturated acids, especially omega-3 PUFA are more prone to oxidation, polymerization and isomerization of double bonds. Distillation at still lower pressures has been used in the isolation of some highly unsaturated acids, and is particularly valuable in polymerization studies to separate monomeric, dimeric and polymeric materials and in the separation of monoacylglycerols from di- and triacylglycerol mixtures.

Another method for making fish oil concentrates is via enzymatic processing, such a lipase catalyzed hydrolysis. The presence of cis carbon-carbon double-bonds in the fatty acids results in bending of the chains. Therefore, the terminal methyl group of the fatty acid lies close to the ester bond which may cause a steric hinderance effect on lipases. The high bending effect of EPA and DHA due to the presence of the 5 and 6 double-bonds, respectively, enhances the steric hinderance effect; therefore, lipases cannot reach the ester-linkage between these fatty acids and glycerol. However, saturated and monounsaturated fatty acids do not present any barriers to lipases and they could be easily hydrolyzed. Therefore, fatty acid selectivity of a lipase for EPA and DHA allows separation and concentration of these fatty acids from others in the remaining portion of marine oils. In addition, lipases have been frequently used to discriminate between EPA and DHA in concentrates containing both of these fatty acids. See, e.g., Bottino, N. R., Vandenberg, G. A. and Reiser, R. (1967) ‘Resistance of Certain Long-chain Polyunsaturated Fatty Acids of Marine Oils to Pancreatic Lipase Hydrolysis’ in Lipids 2, 489-493. In most commercial processes, the hydrolysis is performed on esters produced from fish oils. The end product is accordingly an ester concentrate.

Other methods used in the art for processing fish oils include low temperature crystallization and treatment with solvents (see, e.g., Brown, L. B. and Kolb, D. X. (1955) ‘Application of Low Temperature Crystallization in the Separation of the Fatty Acids and their Compounds’ in Prog. Chem. Fats Lipids 3, 57-94; WO91/13957) and supercritical fluid extraction (see, e.g, Mishra, V. K., Temelli, F. and Ooraikul, B. (1993) ‘Extraction and Purification of Omega 3-Fatty Acids with an Emphasis on Supercritical Fluid Extraction, a Review’ in Food Res. Inter. 26, 217-226).

The concentrated esters produced by these processes can be encapsulated and sold, or the esters can be used to make triglycerides. The TAG form of PUFA is considered to be nutritionally more favorable than methyl or ethyl esters of fatty acids because experimental results have shown impaired intestinal absorption of methyl or ethyl esters of omega-3 PUFA in laboratory animals. Hamazaki, T., Hirai, A., Terano, T., Sajiki, J., Kondo, S., Fujita, T., Tamura, Y. and Kumagai, A. (1982) ‘E ect of Orally Administrated Ethyl Ester of Eicosapentaenoic Acid on PGI-like Substance Production by Rat Aorta’ in Prostaglandins 23, 557-567; El-Boustani, S., Colette, C, Monnier, L., Descomps, B., Paulet, C. A. and Mendey, F. (1987) ‘Eternal Absorption in Man of Eicosapentaenoic Acid in Different Chemical Forms’ in Lipids 22, 711-714; Lawson, L. D. and Hughes, B. G. (1988) ‘Human Absorption of Fish Oil Fatty Acids as Triacylglycerols, Free Fatty Acids or Ethyl Esters’ in Biochem. Biophys. Res. Comm. 152, 328±335. Yang et al. have shown that methyl and ethyl esters are hydrolysed slower than their corresponding TAG. Yang, L. Y., Kuksis, A. and Myher, J. J. (1989) lumenal Hydrolysis of Menhaden and Rapeseed Oils and their Fatty Acid Methyl and Ethyl Esters in the Rats' in Biochem. Cell Biol. 67, 192-204. From a marketing point of view, triacylglycerols of PUFA are often promoted as being more ‘natural’ than other fatty acid derivatives. For these reasons, the esters produced by the processes described above are converted into fatty acids and then incorporated into glycerol by direct esterification or incorporated into glycerides by transesterification. See, e.g., Osada, K., Nakamura, M., Nonaka, M. and Hatano, M. (1992)′Esterification of Glycerol with EPA and DHA by Chromobacterium viscosum and Candida cylindracea Lipases' in J. Jpn. Oil Chem. Soc. 41, 39-43; He, Y. and Shahidi, F. (1997) ‘Enzymatic Esterification of Omega-3-fatty Acid Concentrates from Seal Blubber Oil with Glycerol’ in J. Am. Oil Chem. Soc. 74, 1133-1136; Akoh, C. C., Jennings, B. H. and Lillard, D. A. (1996) ‘Enzymatic Modification of Evening Primrose Oil: Incorporation of n-3 Polyunsaturated Fatty Acids’ in J. Am. Oil Chem. Soc. 73, 1059-1062; Akoh, C. C., Jennings, B. H. and Lillard, D. A. (1995) ‘Enzymatic Modification of Triolein: Incorporation of n-3 Polyunsaturated Fatty Acids’ in J. Am. Oil Chem. Soc. 72, 1317-1321; Sridar, R. and Laksminarayana, G. (1992) ‘Incorporation of Eicosapentaenoic and Docosahexaenoic Acids into Ground Nut Oil by Lipase-catalyzed Ester Exchange’ in J. Am. Oil Chem. Soc. 69, 1041-1042. Basheer, S., Mogi, K. and Nakajuma, M. (1995) ‘Interesterification Kinetics of Triacylglycerides and Fatty Acids with Modified Lipase in n-hexane’ in J. Am. Oil Chem. Soc. 72, 511-518.

Krill oil differs from fish oil in that krill oil comprises high amounts of phospholipids. See e.g., WO 2008/117062; US PUBL. NO. 20080274203. One of the main advantages of krill oil as compared to fish oil is increased bioavailability of omega-3 PUFA in the form of a phospholipid. However, the enzymatic processes described above are not amenable for use with phospholipids. In particular, conversion of esterified or non-esterified omega-3 PUFA back to the phospholipid form is not trivial. Thus, the methods that have been developed for production of fish oil concentrates are not easily transferred to krill oil processing.

Oils with increased amounts of EPA and DHA are desirable because a lower dose is needed to provide the same amount of DHA and EPA. Krill oil concentrates containing increased amounts of omega-3 PUFA in the phospholipid form as compared to other fatty acids in the krill oil compositions have not been developed due to the problems described above. This has been a disadvantage in the market because the fish oil concentrates contain higher amounts of omega-3 PUFA, in particular EPA and DHA, than commercial available krill oil.

Accordingly, what is needed in the art are krill oil concentrates comprising higher amounts of omega-3 PUFA as compared to commercially available krill oils.

### SUMMARY OF THE INVENTION

The present invention relates to krill oil, and in particular to krill oil with elevated levels of omega-3 fatty acids and decreased levels of saturated fatty acids.

In some embodiments, the present invention provides a krill oil, such as a krill oil concentrate, comprising greater than about 22% EPA (w/w total fatty acids), greater than about 10% DHA (w/w total fatty acids), from 4% to 8% myristic acid (w/w total fatty acids), from 3% to 9% c9 oleic acid (w/w total fatty acids), and 20 to 4000 ppm astaxanthin. In some embodiments, the krill oil further comprises about 22% to 30% EPA. In some embodiments, the krill oil further comprises about 10% to 15% DHA. In some embodiments, the krill oil is extracted from Euphausia superba. In some embodiments, the ratio of DHA and EPA:omega 6 (w/w total fatty acids) is from about 10:1 to 14:1. In some embodiments, the ratio of DHA and EPA:c9 oleic acid (w/w total fatty acids) is from about 4:1 to 8:1. In some embodiments, the ratio of DHA and EPA:myristic acid (w/w total fatty acids) is from about 4:1 to 8:1. In some embodiments, the ratio of DHA and EPA:myristic acid and c9 oleic acid (w/w total fatty acids) is from about 2:1 to 4:1. In some embodiments, the ratio omega 3:omega 6 (w/w total fatty acids) is from about 11:1 to 15:1. In some embodiments, the ratio of omega 3:c9 oleic acid (w/w total fatty acids) is from about 5:1 to 9:1. In some embodiments, the ratio of omega 3:myristic acid (w/w total fatty acids) is from about 5:1 to 9:1. In some embodiments, the ratio of omega 3:myristic acid and c9 oleic acid (w/w total fatty acids) is from about 2.5:1 to 4.5:1.

In some embodiments, the present invention provides a capsule containing a krill oil as described above. In some embodiments, the present invention provides a food product containing a krill oil as described above. In some embodiments, the present invention provides a dietary supplement containing a krill oil as described above. In some embodiments, the present invention provides a oil in water emulsion containing a krill oil as described above.

In some embodiments, the krill oils are used for oil administration to a subject. In some embodiments, the krill oil are used for treatment of a condition for which omega-3 is effective.

### DEFINITIONS

As used herein, “krill oil” refers to an oil extracted from Euphausia sp., for example, Euphausia superba.

As used herein, “phospholipid” refers to an organic compound having the following general structure:

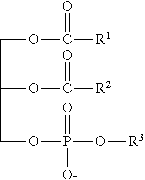

wherein R1 is a fatty acid residue, R2 is a fatty acid residue or —OH, and R3 is a —H or nitrogen containing compound choline (HOCH₂CH₂N⁺(CH₃)₃OH⁻), ethanolamine (HOCH₂CH₂NH₂), inositol or serine. R1 and R2 cannot simultaneously be OH. When R3 is an —OH, the compound is a diacylglycerophosphate, while when R3 is a nitrogen-containing compound, the compound is a phosphatide such as lecithin, cephalin, phosphatidyl serine or plasmalogen.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term omega-6 fatty acid refers to polyunsaturated fatty acids that have the final double bond in the hydrocarbon chain between the sixth and seventh carbon atoms from the methyl end of the molecule.

As used herein, astaxanthin refers to the following chemical structure:

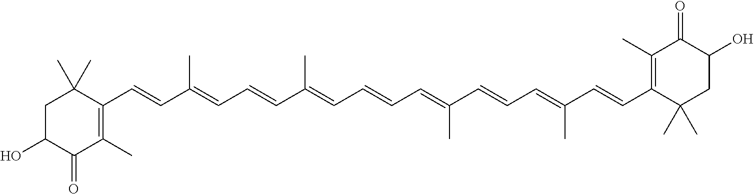 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

As used herein, astaxanthin esters refer to the fatty acids esterified to OH group in the astaxanthin molecule.

As used herein, the term w/w (weight/weight) refers to the amount of a given substance in a composition on weight basis and can be expressed as a percentage. For example, a composition comprising 50% w/w phospholipids means that the mass of the phospholipids is 50% of the total mass of the composition (i.e., 50 grams of phospholipids in 100 grams of the composition, such as an oil). The w/w may also be used to refer to the amount, on a weight basis, of one member of a class of molecules in a composition as compared to all members of the class of molecules. For example, the amount of a particular fatty acid (or class of fatty acids such as omega 3 fatty acids) may be expressed as a percentage of all other fatty acids in the composition on a weight/weight basis, i.e., the weight of the specific fatty acids as a percentage of the total weight of fatty acids in the composition.

### DESCRIPTION OF THE INVENTION

The present invention relates to krill oil, and in particular to krill oil with elevated levels of omega-3 fatty acids and decreased levels of saturated fatty acids. While it was previously known that krill oil contains both phospholipid and triglyceride fractions, the inventors have discovered that krill oil is a multiphase dispersion of these fractions. This property of krill oil has not been previously described. The present inventors have taken advantage of this novel observation to develop processes for separation of the phases based on solubility of phospholipids in a polar solvent. By these processes, it is possible to separate, at least partially, the triglyceride and phospholipid phases. Unexpectedly, analysis of the phospholipid phase has revealed that the phospholipid phase has a higher content of omega-3 PUFA as compared to the triglyceride phase or to commercially available krill oil. The novel krill oil compositions and processes are described below.

### 1. Krill Oil Compositions

In some embodiments, the present invention provides novel krill oil compositions. The novel krill oil compositions are preferably defined by the amount or ratio of total omega-3 PUFA, the amount of EPA and DHA alone or combined, the amount of c9 oleic acid, and/or the amount of myristic acid as compared to previously described krill oils. In some embodiments, the krill oil is produced in whole or in part from Euphausia superba.

In some embodiments, the krill oil comprises greater than about 40% total omega-3 PUFA w/w total fatty acids. By this it is meant that the total amount of omega-3 fatty acids in the krill oil is greater than about 40% of the total fatty acid content of the krill oil on a weight basis. In some of the embodiments described herein, the amounts of particular fatty acids in the krill oil are preferably determined by gas chromatography of a fatty acid methyl esters prepared from the krill oil. In some embodiments, the krill oil comprises greater than about 41%, 42% or 43% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 45% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 46% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 47% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 48% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 49% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 50% total omega-3 PUFA w/w total fatty acids. In some embodiments, the krill oil comprises greater than about 35%, 36%, 37%, 38%, 39%, 40%, 41%, 42% or 43% total omega-3 PUFA w/w total fatty acids up to about 55% total omega-3 PUFA w/w total fatty acids. In some embodiments, the rill oil comprises astaxanthin. In some embodiments, the krill comprises from about 10, 20, 30, 40, 50, 60, 70, 80 or 100 ppm astaxanthin up to about 200, 400, 600, 800, 1000, 1500, 2000, or 4000 ppm astaxanthin. In some embodiments, the krill oil comprises from about 4% to about 8% myristic acid (w/w total fatty acids), from 5% to 7% myristic acid (w/w total fatty acids), or from about 6.0% to 6.6% myristic acid. In some embodiments, the krill oil comprises from about 3% to about 9% c9 oleic acid (w/w total fatty acids), from about 4% to about 8% c9 oleic acid (w/w total fatty acids), from about 5% to about 7% c9 oleic acid (w/w total fatty acids), or from about 6.0% to about 6.6% c9 oleic acid (w/w total fatty acids). In some embodiments, the krill oil comprises less than about 5.0%, 4.5%, 4.0%, 3.8% or 3.6% palmitoleic acid (C16:1; w/w total fatty acids). In some embodiment, the krill oil comprises from about 1% to about 5.0%, about 1.5% to about 4.5%, about 2% to about 4.0%, or about 2.5% to about 3.8% palmitoleic acid (C16:1; w/w total fatty acids).

In some embodiments, the krill oil of the present invention comprises greater than about 22% EPA (w/w total fatty acids), greater than about 10% DHA (w/w total fatty acids), or greater than about 32% EPA and DHA (w/w total fatty acids). In some embodiments, the krill oil of the present invention comprises greater than about 23% EPA (w/w total fatty acids), greater than about 11% DHA (w/w total fatty acids), or greater than about 34% EPA and DHA (w/w total fatty acids). In some embodiments, the krill oil of the present invention comprises greater than about 24% EPA (w/w total fatty acids), greater than about 12% DHA (w/w total fatty acids), or greater than about 36% EPA and DHA (w/w total fatty acids). In some embodiments, the krill oil of the present invention comprises greater than about 25% EPA (w/w total fatty acids), greater than about 12.3% DHA (w/w total fatty acids), or greater than about 37.3% EPA and DHA (w/w total fatty acids). In some embodiments, the krill oil of the present invention comprises greater than about 25.5% EPA (w/w total fatty acids), greater than about 12.5% DHA (w/w total fatty acids), or greater than about 38% EPA and DHA (w/w total fatty acids). In some embodiments, the krill oil comprises an upper limit of 27% EPA and 13% DHA (w/w total fatty acids; total of 40% EPA and DHA), 28% EPA and 14% DHA (w/w total fatty acids; total of 42% EPA and DHA), 30% EPA and 16% DHA (w/w total fatty acids; total of 46% EPA and DHA), 32% EPA and 18% DHA (w/w total fatty acids; total of 50% EPA and DHA), or 37% EPA and 23% DHA (w/w total fatty acids; total of 60% EPA and DHA). In some embodiments, the rill oil comprises astaxanthin. In some embodiments, the krill comprises from about 10, 20, 30, 40, 50, 60, 70, 80 or 100 ppm astaxanthin up to about 200, 400, 600, 800, 1000, 1500 or 2000 ppm astaxanthin. In some embodiments, the krill oil comprises from about 4% to about 8% myristic acid (w/w total fatty acids), from 5% to 7% myristic acid (w/w total fatty acids), or from about 6.0% to 6.6% myristic acid. In some embodiments, the krill oil comprises from about 3% to about 9% c9 oleic acid (w/w total fatty acids), from about 4% to about 8% c9 oleic acid (w/w total fatty acids), from about 5% to about 7% c9 oleic acid (w/w total fatty acids), or from about 6.0% to about 6.6% c9 oleic acid (w/w total fatty acids).

In some embodiments, the krill oil has a ratio of DHA and EPA:omega 6 PUFAs (w/w total fatty acids) of from about 10:1 to 14:1, 11:1 to 13:1, 11.3:1 to 12.1:1, or 11.5:1 to 11.9:1. In some embodiments, the krill oil has a ratio of DHA and EPA:c9 oleic acid (w/w total fatty acids) of from about 4:1 to 8:1, 5:1 to 7:1, 5.7:1 to 6.9:1, or 6.0:1 to 6.6:1. In some embodiments, the krill oil has a ratio of DHA and EPA:myristic acid (w/w total fatty acids) of from about 4:1 to 8:1, 5:1 to 7:1, 5.7:1 to 6.9:1, or 6.0:1 to 6.6:1. In some embodiments, the krill oil has a ratio of DHA and EPA:myristic acid and c9 oleic acid (w/w total fatty acids) of from about 2:1 to 4:1, 2.4:1 to 3.5:1, 2.7:1 to 3.5:1, or 2.9:1 to 3.3:1.

In some embodiments, the krill oil has a ratio omega-3 PUFAs:omega 6 PUFAs (w/w total fatty acids) of from about 11:1 to 15:1, 12:1 to 14:1, 12.5:1 to 13.5:1, or 12.8:1 to 13.2:1. In some embodiments, the krill oil has a ratio omega-3 PUFAs:c9 oleic acid (w/w total fatty acids) of from about 5:1 to 9:1, 6:1 to 8:1, 6.2:1 to 7.4:1, or 6.4:1 to 7.2:1. In some embodiments, the krill oil has a ratio omega-3 PUFAs:myristic acid (w/w total fatty acids) of from about 5:1 to 9:1, 6:1 to 8:1, 6.2:1 to 7.4:1, or 6.4:1 to 7.2:1. In some embodiments, the krill oil has a ratio omega-3 PUFAs:myristic acid and c9 oleic acid (w/w total fatty acids) of from about 2:1 to 5:1, 2.5:1 to 4.5:1, 3:1 to 3.9:1, or 3.2:1 to 3.6:1.

In some embodiments, the krill oil of this invention is formulated with acceptable excipients and/or carriers for oral consumption. The actual form of the carrier, and thus, the composition itself, is not critical. The carrier may be a liquid, gel, gelcap, capsule, powder, solid tablet (coated or non-coated), tea, or the like. The composition is preferably in the form of a tablet or capsule and most preferably in the form of a soft gel capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In some embodiments, dietary supplements of the present invention comprise krill oil as described above and one or more inert ingredients, especially if it is desirable to limit the number of calories added to the diet by the dietary supplement. The dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In further embodiments, the compositions comprise at least one food flavoring such as acetaldehyde (ethanal), acetoin (acetyl methylcarbinol), anethole (parapropenyl anisole), benzaldehyde (benzoic aldehyde), N butyric acid (butanoic acid), d or l carvone (carvol), cinnamaldehyde (cinnamic aldehyde), citral (2,6 dimethyloctadien 2,6 al 8, gera nial, neral), decanal (N decylaldehyde, capraldehyde, capric aldehyde, caprinaldehyde, aldehyde C 10), ethyl acetate, ethyl butyrate, 3 methyl 3 phenyl glycidic acid ethyl ester (ethyl methyl phenyl glycidate, strawberry aldehyde, C 16 aldehyde), ethyl vanillin, geraniol (3,7 dimethyl 2,6 and 3,6 octadien 1 ol), geranyl acetate (geraniol acetate), limonene (d, l, and dl), linalool (linalol, 3,7 dimethyl 1,6 octadien 3 ol), linalyl acetate (bergamol), methyl anthranilate (methyl 2 aminobenzoate), piperonal (3,4 methylenedioxy benzaldehyde, heliotropin), vanillin, alfalfa (Medicago sativa L.), allspice (Pimenta officinalis), ambrette seed (Hibiscus abelmoschus), angelic (Angelica archangelica), Angostura (Galipea officinalis), anise (Pimpinella anisum), star anise (Illicium verum), balm (Melissa officinalis), basil (Ocimum basilicum), bay (Laurus nobilis), calendula (Calendula officinalis), (Anthemis nobilis), capsicum (Capsicum frutescens), caraway (Carum carvi), cardamom (Elettaria cardamomum), cassia, (Cinnamomum cassia), cayenne pepper (Capsicum frutescens), Celery seed (Apium graveolens), chervil (Anthriscus cerefolium), chives (Allium schoenoprasum), coriander (Coriandrum sativum), cumin (Cuminum cyminum), elder flowers (Sambucus canadensis), fennel (Foeniculum vulgare), fenugreek (Trigonella foenum graecum), ginger (Zingiber officinale), horehound (Marrubium vulgare), horseradish (Armoracia lapathifolia), hyssop (Hyssopus officinalis), lavender (Lavandula officinalis), mace (Myristica fragrans), marjoram (Majorana hortensis), mustard (Brassica nigra, Brassica juncea, Brassica hirta), nutmeg (Myristica fragrans), paprika (Capsicum annuum), black pepper (Piper nigrum), peppermint (Mentha piperita), poppy seed (Papayer somniferum), rosemary (Rosmarinus officinalis), saffron (Crocus sativus), sage (Salvia officinalis), savory (Satureia hortensis, Satureia montana), sesame (Sesamum indicum), spearmint (Mentha spicata), tarragon (Artemisia dracunculus), thyme (Thymus vulgaris, Thymus serpyllum), turmeric (Curcuma longa), vanilla (Vanilla planifolia), zedoary (Curcuma zedoaria), sucrose, glucose, saccharin, sorbitol, mannitol, aspartame. Other suitable flavoring are disclosed in such references as Remington's Pharmaceutical Sciences, 18th Edition, Mack Publishing, p. 1288-1300 (1990), and Furia and Pellanca, Fenaroli's Handbook of Flavor Ingredients, The Chemical Rubber Company, Cleveland, Ohio, (1971), known to those skilled in the art.

In other embodiments, the compositions comprise at least one synthetic or natural food coloring (e.g., annatto extract, astaxanthin, beet powder, ultramarine blue, canthaxanthin, caramel, carotenal, beta carotene, carmine, toasted cottonseed flour, ferrous gluconate, ferrous lactate, grape color extract, grape skin extract, iron oxide, fruit juice, vegetable juice, dried algae meal, tagetes meal, carrot oil, corn endosperm oil, paprika, paprika oleoresin, riboflavin, saffron, tumeric, tumeric and oleoresin).

In still further embodiments, the compositions comprise at least one phytonutrient (e.g., soy isoflavonoids, oligomeric proanthcyanidins, indol 3 carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, cathechins, gallates, and quercitin). Sources of plant phytonutrients include, but are not limited to, soy lecithin, soy isoflavones, brown rice germ, royal jelly, bee propolis, acerola berry juice powder, Japanese green tea, grape seed extract, grape skin extract, carrot juice, bilberry, flaxseed meal, bee pollen, ginkgo biloba, primrose (evening primrose oil), red clover, burdock root, dandelion, parsley, rose hips, milk thistle, ginger, Siberian ginseng, rosemary, curcumin, garlic, lycopene, grapefruit seed extract, spinach, and broccoli.

In still other embodiments, the compositions comprise at least one vitamin (e.g., vitamin A, thiamin (B1), riboflavin (B2), pyridoxine (B6), cyanocobalamin (B12), biotin, ascorbic acid (vitamin C), retinoic acid (vitamin D), vitamin E, folic acid and other folates, vitamin K, niacin, and pantothenic acid). In some embodiments, the particles comprise at least one mineral (e.g., sodium, potassium, magnesium, calcium, phosphorus, chlorine, iron, zinc, manganese, flourine, copper, molybdenum, chromium, selenium, and iodine). In some particularly preferred embodiments, a dosage of a plurality of particles includes vitamins or minerals in the range of the recommended daily allowance (RDA) as specified by amino acid supplement formula in which at least one amino acid is included (e.g., 1-carnitine or tryptophan).

In some embodiments, the present invention provides functional food products containing krill oil as described above. Examples of functional foods include, but are not limited to dairy products such yogurt, milk and cheese, cereals, beverages, shakes, powdered supplements, and the like.

### 2. Processes for Making Krill Oil

The processes of the present invention are useful with krill oil produced by a variety of processes. Suitable processes for producing krill oil include extraction with polar solvents such as ethanol, supercritical fluid extraction, extraction with non-polar organic solvents such as acetone, cold pressing, etc. See, e.g., WO2009/027692, WO2008/117062, WO2003/011873, all of which are incorporated herein by reference. The processes of the present invention may also be performed on commercially available krill oils such as those supplied by Aker Biomarine, Neptune Bioressources, and Enzymotec.

As described above, the present inventors have discovered that krill oil is a multiphase dispersion. The present invention provides processes for separating the multiphase dispersion into two or more phases that can be separated. In some embodiments, krill oil in the multiphase dispersion state is further processed by mixing the krill oil with a polar solvent and incubating the mixture for a period of time (the incubation period) sufficient for the formation of least two phases in the mixture. The upper phase, or phospholipid phase, comprises the krill oil of the present invention.

In preferred embodiments, the phospholipid phase is separated from any other phases formed during the incubation phase, for example, by decanting the phospholipid phase. In some embodiments, the incubation phase is from about 0.5 hours to about 48 hours, 0.5 hours to 24 hours, 0.5 hours to 12 hours, 0.5 hours to 6 hours, 0.5 hours to 4 hours, or 1 hour to 4 hours. In some embodiments, the incubation is conducted at from about 0 C to about 25 C. In some embodiments, the incubation is conducted at from about 4 C to about 10 C, about 4 C to about 20 C, about 4 C to about 25 C, about 10 C to about 20 C, about 10 C to about 25 C, or about 15 C to 25 C.

In some embodiments, the polar solvent is an alcohol, such as a monohydric alcohol. Suitable monohydric alcohols include, but are not limited to, methanol, ethanol, propanol and isopropanol. Other polar solvents include dimethyl sulfoxide (DMSO), formamide, acetonitrile, N,N-dimethylformamide (DNF) and other solvents with a dielectric constant of higher than 15 or 20. In some embodiments, the krill oil is diluted with the polar solvent at a ratio of krill oil:polar solvent of 1:0.5 to 1:10, 1:1 to 1:5, 1:1 to 1:3, 1:2 to 1:5, 1:2 to 1:4, or 1:2 to 1:3. In some embodiments, the polarity of the solvent is adjusted by adding water. In some embodiments, the ratio of polar solvent, for example ethanol, to water is from about 1:1 to 100:1, 2:1 to 100:1, 2:1 to 20:1, 3:1 to 20:1, 4:1 to 20:1, 5:1 to 20:1, or 10:1 to 20:1.

### 3. Uses of Krill Oil

The krill oil of the present invention is useful for treatment of any disease, disorder or condition in which omega-3 PUFAs have been shown to be effective. Diseases and disorders that may be treated with the omega-3 fatty acid formulations described herein include alopecia, Alzheimer's dementia, angina, anxiety disorders, asthma, attention deficit disorder, attention-deficit hyperactivity disorder, atopic dermatitis, autism, bipolar disorder, borderline personality disorder, cardiovascular disease, chronic fatigue syndrome, chronic pain, chronic polyarthritis, cognitive disorders, communication disorders, colitis, Crohn's disease, cystic fibrosis, dementia, depression, diabetes (of the non-insulin dependent or insulin dependent forms), diabetes-related sequelae, diabetic neuropathy, dry eyes and other inflammatory eye disorders, dry skin, dysmenorrhea, eating disorders (such as anorexia nervosa or bulimia nervosa and obesity), eczema, fibromyalgia, gout, learning disorders (e.g. reading, spelling, mathematics, receptive, and expressive language, and motor skills disorders), lupus, male infertility, metabolic syndrome, melanoma, mild cognitive impairment, migraine, mood disorders, multiple sclerosis, obsessive-compulsive disorder, oppositional-defiant disorder, osteoarthritis, osteoporosis, pervasive developmental disorders, polyarteritis nodosa, psoriasis, psoriatic arthritis, rheumatoid arthritis, schizophrenia, sclerodermia, self-injurious behavior, sickle cell anemia, tic disorders, tinnitus, ulcerative colitis, or vasculitic disorders (such as polyarteritis nodosa and temporal arthritis. Cardiovascular disease and disorders that can be treated with the omega-3 fatty acid formulations described herein include angina, atherosclerosis, hypercholesterolemia, hypertriglyceridemia, low HDL, high blood pressure, Raynaud's disease, and cardiac arrhythmias. Methods of treatment with the omega-3 fatty acid formulations described herein include prophylaxis with Omega-3 formulations to prevent post-cardiotomy (including but not limited to coronary artery bypass graft surgery and valve surgery) complications (including but not limited to depression, neuro-cognitive decline, congestive heart failure and infarction, clotting events, and arrhythmias) as well as for the treatment for such complications.

### EXPERIMENTAL

### Example 1

Krill oil is extracted from krill meal (Aker Biomarine) by ethanol extraction. Briefly, krill meal is extracted with ethanol for 1 hour at 15-30 C. The liquid fraction is separated by filtration. The liquid fraction is concentrated by evaporating the ethanol under a vacuum at about 50 C until the concentration of ethanol is reduced to about 20%. The concentrated liquid phase is then centrifuged to remove any remaining solids or precipitates and then evaporated under a vacuum to a final concentration of less than 0.5% ethanol. The extracted krill oil is a multiphase dispersion. An exemplary batch of krill oil prepared by this process had the following composition.

| | | |
|---|---|---|
| Triacylgycerol | g/100 g oil | 30 |
| Diacylglycerol | g/100 g oil | 0.7 |
| Monoacylglycerol | g/100 g oil | <1 |
| Free fatty acids | g/100 g oil | 4.8 |
| Cholesterol | g/100 g oil | 1.2 |
| Cholesterol esters | g/100 g oil | <0.5 |
| Phosphatidylethanolamine | g/100 g oil | 1.5 |
| Phosphatidylinositol | g/100 g oil | <1 |
| Phosphatidylserine | g/100 g oil | <1 |
| Phosphatidylcholine | g/100 g oil | 40 |
| Lyso-phosphatidylcholine | g/100 g oil | 3.3 |
| Total polar lipids | g/100 g oil | 44.6 |
| Total neutral lipids | g/100 g oil | 36.9 |
| Total sum lipids | g/100 g oil | 81.5 |

### Example 2

This example describes attempts to concentrate omega-3 PUFA in krill oil by lowering temperature. It was thought that by lowering the temperature, highly saturated fat would turn solid at a faster rate and sediment or in another way form a layer in the column of krill oil. The experiment tested whether triacylglycerol (TAG) and phospholipids (PL) with saturated fatty acyl chains would form a layer or if TAG and PL would form layers independent of the fatty acyl chain type. Krill oil described in Example 1 was diluted with 20% absolute ethanol and a glass column was filled. The column was placed in a refrigerator overnight. The oil was examined for the formation of layers. The oil turned very viscous, paler but not white. No layers formed. The oil was then taken to room temperature. Again, there was no layer formation. It was not possible to determine if some portion of the fat turns solid faster than other parts. The lack of layer formation can possibly be explained by the fact that the viscosity of the oil is too high to allow vertical movement of fat with higher or lower density. Dilution with 50% absolute ethanol had the same result of no layer formation even though the solution was less viscous.

### Example 3

This example describes the concentration of omega-3 PUFA in krill oil. Krill oil described in Example 1 was diluted 1:1 with absolute ethanol (B), 1:2 with absolute ethanol (C), 1:3 with absolute ethanol (D), and 1:3 with 95% ethanol (E) in 15 ml polypropylene vials. No phase separation was seen in B, C, or D. E was slightly opaque and after five minutes a layer formed at the bottom. All four vials were then stored at −30 C for two hours. At this time, the oil was solidified and appeared as a white solid. No visible layers were observed in the solid form. After thawing at room temperature, the solid oil melted and drops formed that sedimented in the vials C, D, and E. The volume of the bottom layer increased with increasing ethanol dilution. Vial E had a larger lower phase than vial D. The bottom layer was darker than the top layer in all vials but C.

The solubility of the lower phase was examined. The lower phase was not soluble in ethanol or water. The lower phase exhibited good solubility in hexane and the color was more brown than the top layer.

The UV spectra of the top and bottom layers was examined. The UV spectra of the lower phase indicated the presence of astaxanthin together with components absorbing at lower wavelengths. The UV spectra of the upper phase indicated a similar pattern with astaxanthin less prominent compared to lower wavelengths.

The upper and lower phases were examined by thin layer chromatography (TLC). The TLC data indicates that after separation of krill oil into two phases is a higher portion of TAG in the lower phase than in the upper phase. It also appears that there is more PL in the upper phase.

The upper and lower phases were next examined by GC-FID. The results are provided below.

| Fraction | Upper | Upper | Lower | Lower |
|---|---|---|---|---|
| ID | 1 (D) | 2 (E) | 3 (D) | 4 (E) |
| C12:0 | 0.1 | 0.2 | 0.4 | 0.4 |
| C14:0 | 6.3 | 6.4 | 17.4 | 17.6 |
| C15:0 | 0.4 | 0.3 | 0.5 | 0.5 |
| C16:0 | 19.9 | 20.1 | 19.6 | 19.8 |
| C16:1 | 3.5 | 3.5 | 7.8 | 7.8 |
| C18:0 | 0.9 | 0.9 | 1.3 | 1.3 |
| C18:1, t6-11 | 0.3 | 0.3 | 0.6 | 0.6 |
| C18:1, c9 | 6.3 | 6.4 | 13.2 | 13.2 |
| C18:1, c11 | 5.7 | 5.7 | 6.8 | 6.7 |
| C18:2, n-6 | 1.6 | 1.6 | 1.8 | 1.8 |
| C20:0 | 0.1 | 0.1 | 0.1 | 0.1 |
| C18:3, n-6 | 0.3 | 0.2 | 0.2 | 0.2 |
| C18:3, n-3 | 1.0 | 1.0 | 0.8 | 0.8 |
| C20:1, n-9 | 0.5 | 0.5 | 0.8 | 0.8 |
| C18:4, n-3 | 2.4 | 2.4 | 3.2 | 3.2 |
| C20:2, n-6 | 0.1 | 0.1 | 0.1 | 0.1 |
| C22:0 | 0.2 | 0.2 | 0.2 | 0.2 |
| C20:3, n-6 | 0.1 | 0.1 | 0.1 | 0.1 |
| C20:3, n-3 | 0.1 | 0.1 | 0.1 | 0.1 |
| C20:4, n-6/C22:1, n-9 | 1.3 | 1.3 | 0.5 | 0.5 |
| C20:4, n-3 | 0.4 | 0.4 | 0.2 | 0.2 |
| C20:5, n-3 | 26.0 | 26.0 | 7.7 | 7.7 |
| C22:5, n-3 | 0.6 | 0.6 | 0.3 | 0.3 |
| C22:6, n-3 | 12.7 | 12.7 | 3.8 | 3.7 |
| SFA | 27.8 | 28.2 | 39.6 | 40.0 |
| MUFA | 16.3 | 16.4 | 29.2 | 29.2 |
| PUFA (n-6) | 3.3 | 3.3 | 2.5 | 2.6 |
| PUFA (n-3) | 43.1 | 43.2 | 16.1 | 16.0 |
| Total PUFA | 46.4 | 46.4 | 18.6 | 18.6 |

This analysis indicates that there is a higher relative portion of omega-3 PUFA in the upper phase compared to the lower phase. The upper phase has 43% total omega-3 PUFA. EPA and DHA is equally concentrated. In the lower phase the fatty acids 14:0 (myristic acid) and 18:1 (c9 oleic acid) are concentrated. There are no changes in 16:0.

This data describes the separation of a krill oil with approximately 40% nonpolar lipids (30% TAG) into two phases by adding ethanol/water and lowering the temperature. The layers form by passive sedimentation. TLC data indicates that PL is high in the upper phase and that TAG is high in the lower phase. GC-FID data shows that omega-3 lipids is high in the upper phase (43%, 43 g/100 g FAME) and 18% in the lower phase. An intriguing observation is that the particularly unhealthy fatty acid 14:0 is lowered in the upper phase.

The present invention is not limited to any particular mechanism. Nevertheless, the inventors have discovered that krill oil produced by ethanol extraction is a multiphase dispersion. Unsoluble components such as triglycerides are entrapped in the soluble phase (polar lipids) and coextracted. The processes describe above take advantage of this fact to provide krill oil compositions with concentrated amounts of desirable omega-3 fatty acids.

1. Krill oil comprising greater than about 22% EPA (w/w total fatty acids), greater than about 10% DHA (w/w total fatty acids), from 4% to 8% myristic acid (w/w total fatty acids), and from 3% to 9% c9 oleic acid (w/w total fatty acids).

2. Krill oil of claim 1, further comprising about 22% to 30% EPA.

3. Krill oil of claim 1, further comprising about 10% to 15% DHA.

4. Krill oil of claim 1, wherein said krill oil is extracted from Euphausia superba.

5. Krill oil of claim 1, wherein the ratio of DHA and EPA:omega 6 (w/w total fatty acids) is from about 10:1 to 14:1.

6. Krill oil of claim 1, wherein the ratio of DHA and EPA:c9 oleic acid (w/w total fatty acids) is from about 4:1 to 8:1.

7. Krill oil of claim 1, wherein the ratio of DHA and EPA:myristic acid (w/w total fatty acids) is from about 4:1 to 8:1.

8. Krill oil of claim 1, wherein the ratio of DHA and EPA:myristic acid and c9 oleic acid (w/w total fatty acids) is from about 2:1 to 4:1.

9. Krill oil of claim 1, wherein the ratio omega 3:omega 6 (w/w total fatty acids) is from about 11:1 to 15:1.

10. Krill oil of claim 1, wherein the ratio of omega 3:c9 oleic acid (w/w total fatty acids) is from about 5:1 to 9:1.

11. Krill oil of claim 1, wherein the ratio of omega 3:myristic acid (w/w total fatty acids) is from about 5:1 to 9:1.

12. Krill oil of claim 1, wherein the ratio of omega 3:myristic acid and c9 oleic acid (w/w total fatty acids) is from about 2.5:1 to 4.5:1.

13. A capsule containing the krill oil of claim 1.

14-18. (canceled)

19. The krill oil of claim 1, further comprising from about 20 to 4000 ppm astaxanthin.
